(19) United States
(12) Patent Application Publication
(10) Pub. No.: US 2025/0208365 A1
(43) Pub. Date: Jun. 26, 2025

(54) OPTICAL FIBRE TERMINATOR

(71) Applicant: The Automation Partnership (Cambridge) Limited, Royston, Hertfordshire (GB)

(72) Inventor: Sullivan SEAN, Baldock, Hertfordshire (GB)

(21) Appl. No.: 18/852,240

(22) PCT Filed: Mar. 31, 2023
(86) PCT No.: PCT/EP2023/058462

§ 371 Date: Sep. 27, 2024

(30) Foreign Application Priority Data
Apr. 5, 2022 (EP) 22166842.9

(51) Int. Cl. G02B 6/44

(52) U.S. Cl. CPC G02B 6/4471

(57) ABSTRACT

The present invention provides an optical fibre terminator (1, la-1d) for terminating two or more parallel optical fibres (4), each optical fibre comprising an optical waveguide core surrounded by a protective polymer jacket. The terminator includes a housing (2) having a front face (3) and a backshell (4). The housing is configured for insertion thereinto of the two or more parallel optical fibres so that respective end faces (5) of the two or more optical fibres are located at predetermined spaced positions relative to the front face and the two or more optical fibres extend rearwardly away from the front face through the backshell. The terminator also includes one or more gripping spacers (6) subsequently insertable into one or more respective matching channels (8) formed in the backshell behind the front face such that each gripping spacer, on its insertion, non-piercingly grips a jacketed flank of at least one optical fibre on a opposite side of the spacer to thereby retain the fibres in the housing with the fibre end faces locked at the predetermined spaced positions.

This application claims priority from EP 22166842.9 filed 5 Apr. 2022, the contents and elements of which are herein incorporated by reference for all purposes.

### FIELD OF THE INVENTION

The present invention relates to an optical fibre terminator.

### BACKGROUND

Cell culture is a process for growing cells in an artificial environment such as a bioreaction vessel. Typically, the cells are grown whilst suspended in a culture growth medium. Monitoring the environment to which the cells are exposed in the bioreaction vessel is important for controlling this environment, and ultimately the physiology of the cells and the amount of target produced. In particular, monitoring of parameters such as DO (amount of dissolved oxygen), pH of the culture growth medium within the bioreaction vessel is key to exerting this control, and pCO₂ (partial pressure of CO₂).

Monitoring of such parameters can be achieved invasively and/or non-invasively. Invasive monitoring generally uses a sensor probe inserted directly into the culture growth medium contained in the vessel. However, this can create sensor calibration difficulties and, as probes are generally re-usable, can increase a risk of cross-contamination between vessels. In contrast, non-invasive monitoring sensors are not placed in direct contact with the culture growth medium, but may, for example, be positioned outside the vessel. While this avoids some of the problems associated with invasive monitoring, it is critical that non-invasive sensors located outside the vessel are arranged correctly in relation to the vessel to properly calibrate the sensor, to avoid drift and also to provide accurate and consistent measurements in line with previous calibrations. In another approach, disposable patch monitoring sensors are placed in the bioreaction vessel below the level of growth medium, e.g. on the a base wall of the vessel. However, these sensors require coupling to external reading devices, and for reliable monitoring, it is critical that the coupling between patch sensor and any reading device is well defined. Thus the reading device, if not in actual close contact with the opposite (outer) face of the vessel wall on which the patch sensor locates, must be positioned within a controlled spacing, which is typically between 0.5 and 0.2 mm from that face.

Further challenges associated with monitoring arise in relation to parallel cell culturing using large numbers of relatively small bioreaction vessels. Such an approach to cell culturing can be used to mimic larger scale cell culture environments and can improve the ratio of success and speed to market of new biopharmaceutical products by enabling screening of large numbers of potential cell clones in small-scale production-like processes. The most suitable clones can then be identified and taken forward to larger-scale development processes. Bioreaction vessels for use in such screening trials are not only small in scale, but typically are tightly arranged together and may be consumable items. However, monitoring parameters of the environment within each small-scale bioreaction vessel imposes constraints on sensor technologies. In particular, when using non-invasive probes, it is necessary to satisfy both stringent probe size limitations while also precisely and securely arranging large numbers of the probes relative to the bioreaction vessels in confined spaces.

Optical fibres comprising an optical waveguide core surrounded by a protective jacket can be used as non-invasive sensor elements to monitor parameters of the cell cultures contained in such multi-vessel bioreactor systems, e.g. via disposable patch monitoring sensors placed in the vessels. However, the jackets are usually made of soft plastic, typically polyurethane or polyethylene, which is not bonded to the optical fibre. This can cause difficulties with locating and robustly securing optical fibres in correct alignment with a vessel for parameter monitoring, as particularly towards the ends of the fibres the jacket can slide relative to the optical waveguide core. To complicate matters further, soft plastics such as polyurethane or polyethylene generally cannot be glued, while piercing the jacket to secure directly to the core can create stress points in the optical waveguide core and cause it to shear.

A known approach to securing optical fibres in a desired position involves threading the fibres through a clamp plate positioned underneath a bioreaction vessel and, when the fibres are located at the desired position for parameter monitoring, tightening retaining screws on the clamp plate. The fibres are thus secured, and risk of damage thereto is mitigated by respective O-rings positioned at each fibre location on the plate, the O-rings deforming under screw tightening to mediate the clamping force between the plate and the fibre. However, such a clamp plate configuration is not generally suitable for small-scale and closely spaced bioreaction vessels. In particular, the O-rings impose a lower limit on the possible spacing between fibres on the clamp plate, which in turn imposes lower limits on the size and spacing of the vessels. Furthermore, the optical fibres can be difficult to replace without access to the retaining screws, making fibre replacement a complicated and time-consuming procedure.

An alternative approach involves removing a portion of the protective jacket of the optical fibre to expose a portion of the optical waveguide core that can then be glued to a suitable mounting material to secure the optical fibre in a desired position. However, this typically requires special glues, materials and tooling, and careful handling procedures as waveguide cores are highly brittle, and further complicates the replacement of damaged or malfunctioning fibres. For these reasons, such an approach is not generally adopted.

Thus, securing optical fibres in a desired position for performing parameter monitoring of cell cultures contained in a small-scale bioreaction vessels presents a significant technical challenge.

### SUMMARY OF THE INVENTION

In general terms, the present invention provides a coupler for coupling parallel elongate members, the coupler including:

a housing having a front face and a backshell, and being configured for insertion thereinto of the two or more parallel elongate members so that respective end faces of the two or more elongate members are located at predetermined spaced positions relative to the front face and the two or more elongate members extend rearwardly away from the front face through the backshell of the housing; and one or more gripping spacers insertable into one or more respective, matching channels formed in the backshell behind the front face such that, on insertion, each gripping spacer grips a jacketed flank of at least one elongate member on a side of the spacer to retain the elongate members in the housing with their end faces locked at the predetermined spaced positions.

Each elongate member can thus be retained in the housing by virtue of being gripped, on one flank, by a gripping spacer and, on an opposite flank, by being consequently pressed against a wall of the housing or against a flank of an adjacent elongate member. By retaining the elongate members in the housing with the gripping spacers, the elongate members can be immobilised both laterally and longitudinally within the housing to lock their end faces at the predetermined spaced positions. Preferably, the or each gripping spacer non-piercingly grips the jacketed flank of the at least one elongate member. As it is an inserted side of the or each gripping spacer that grips its respective jacketed flank, the act of insertion of the spacer itself leads to the elongate member being gripped, which helps to simplify the process of mounting of the elongate members in the coupler.

As an example, the coupler may be for coupling two parallel elongate members, the coupler including:
a housing having a front face and a backshell, and being configured for insertion thereinto of the two parallel elongate members so that respective end faces of the two elongate members are located at predetermined spaced positions relative to the front face and the two elongate members extend rearwardly away from the front face through the backshell; and
a gripping spacer insertable into a matching channel formed in the backshell behind the front face and between the predetermined spaced positions such that, on insertion, the gripping spacer grips jacketed flanks of the two elongate members on opposite sides of the spacer to thereby space the elongate members apart and retain the elongate members in the housing with their end faces locked at the predetermined spaced positions.

The elongate members can be wires, tubes or pipes, for example, but more particularly the elongate members can be optical fibres, and the coupler can be a terminator for terminating the optical fibres.

In a first aspect, the present invention provides an optical fibre terminator for terminating two or more parallel optical fibres, each optical fibre comprising an optical waveguide core surrounded by a protective polymer jacket, the terminator including:
a housing having a front face and a backshell, and being configured for insertion thereinto of the two or more parallel optical fibres so that respective end faces of the two or more optical fibres are located at predetermined spaced positions relative to the front face and the two or more optical fibres extend rearwardly away from the front face through the backshell of the housing; and
one or more gripping spacers insertable into one or more respective, matching channels formed in the backshell behind the front face such that, on insertion, each gripping spacer grips a jacketed flank of at least one optical fibre on a side of the spacer to thereby retain the fibres in the housing with the fibre end faces locked at the predetermined spaced positions.

Each optical fibre can thus be retained in the housing by virtue of being gripped, on one flank, by a gripping spacer and, on an opposite flank, by being consequently pressed against a wall of the housing or against a flank of an adjacent elongate member. By retaining the optical fibres in the housing with the gripping spacers, the optical fibres can be immobilised both laterally and longitudinally within the housing to lock their end faces at the predetermined spaced positions. Preferably, the or each gripping spacer non-piercingly grips the jacketed flank of the at least one optical fibre. As it is an inserted side of the or each gripping spacer that grips its respective jacketed flank, the act of insertion of the spacer itself leads to the fibre being gripped, which helps to simplify the process of mounting of the fibres in the terminator.

As a first example of the first aspect, the present invention provides an optical fibre terminator for terminating two parallel optical fibres, each optical fibre comprising an optical waveguide core surrounded by a protective polymer jacket, the terminator including:
a housing having a front face and a backshell, and being configured for insertion thereinto of the two parallel optical fibres so that respective end faces of the two optical fibres are located at predetermined spaced positions relative to the front face and the two optical fibres extend rearwardly away from the front face through the backshell; and
a gripping spacer insertable into a matching channel formed in the backshell behind the front face and between the predetermined spaced positions such that, on insertion, the gripping spacer grips jacketed flanks of the two optical fibres on opposite sides of the spacer to thereby space the fibres apart and retain the fibres in the housing with the fibre end faces locked at the predetermined spaced positions.

As a second example of the first aspect, the present invention provides an optical fibre terminator for terminating two parallel optical fibres, each optical fibre comprising an optical waveguide core surrounded by a protective polymer jacket, the terminator including:
a housing having a front face and a backshell, and being configured for insertion thereinto of the two parallel optical fibres so that respective end faces of the two optical fibres are located at predetermined adjacent positions relative to the front face and the two optical fibres extend rearwardly away from the front face through the backshell; and
two gripping spaces are insertable into respective matching channels formed in the backshell behind the front face and to substantially opposing sides of the predetermined adjacent positions such that, on insertion, each gripping spacer grips a jacketed flank of a respective one of the optical fibres to thereby urge the fibres together and retain the fibres in the housing with the fibre end faces locked at the predetermined adjacent positions.

In this example, the retained fibres can be urged together with flanks of the two fibres pressed into direct contact with each other, or urged together with flanks of the two fibres pressed into contact with a separator provided by the housing which interposes between the two fibres to impose a fixed spacing on them. Advantageously, the terminator of the first aspect can be easy to assemble to the fibres, and requires no gluing or bonding to connect the optical fibres, thereby facilitating replacement of individual damaged or malfunctioning fibres. Moreover, the terminator does not require the polymer jackets to be pierced in order to grip the fibres and therefore reduces the risks of structural damage to the optical waveguide cores. Rather, the terminator can firmly and safely retain the fibres via the gripping spacer(s) e.g. to prevent any unintentional withdrawal of the fibres from the housing. Thus, the terminator reduces costs and increases reliability compared to conventional fibre attachment solutions discussed above. Additionally, the present terminator can be made compact and is thus useable even with small-scale and closely spaced bioreaction vessels.

The or each gripping spacer may have a serrated side to grip the jacketed flank of the respective inserted fibre. Advantageously, the opposite side can increase the resistance to fibre withdrawal provided by the gripping spacer, e.g. by biting onto the jacketed flank of the fibre, thereby improving the fibre retention within the housing. In the first example above, the gripping spacer may have serrated opposite sides to grip the jacketed flanks of the inserted fibres. In this case, both the serrated opposite sides can increase the resistance to fibre withdrawal provided by the gripping spacer, e.g. by biting onto the jacketed flanks of the fibres, thereby improving the fibre retention within the housing.

Another option is for the or each gripping spacer to be made of a rubber or elastomeric material that is pulled into its matching channel, the pulling stretching the material and causing the gripping spacer to contract transversely so that, when released to its unstretched state, the gripping spacer expands transversely to grip the jacket and retain it by frictional interaction therewith.

The or each gripping spacer may be removably insertable. Advantageously, this can facilitate the replacement of damaged or malfunctioning fibres. For example, to replace a fibre, the or a spacer may be removed such that it no longer retains its fibre(s) in the housing, and a selected fibre can then easily be removed by extracting it from the terminator. Similarly, a replacement fibre can then be inserted in the same position and the fibres re-secured by re-inserting the gripping spacer, e.g. between the fibres in the case of the first example above.

The or each gripping spacer may have an obround cross section perpendicular to its insertion direction, the sides of the spacer(s) which grip the jacketed flanks of the optical fibres being the straight edges of the obround cross section(s). Advantageously, this shape can provide a relatively large contact area between the gripping spacer and its jacketed fibre(s) to spread loads over the jacketed flanks and reduce risk of damage to the fibres.

The front face of the housing may include respective openings to receive the fibre end faces and to define the predetermined spaced positions. For example, the optical fibre terminator may be configured such that when the fibre end faces are located at the predetermined spaced positions, the fibre end faces are flush with the front face of the housing. Advantageously, this can ensure a close contact between the fibre end faces and a wall of a bioreaction vessel, which can in turn reduce a risk of compromised signal phase and amplitude and/or loss of control during measurements when using the fibres as non-invasive probes.

The optical fibre terminator may be a duplex fibre terminator for terminating just two parallel optical fibres. For example, a duplex fibre terminator may connect to a first optical fibre for monitoring pH levels of a cell culture and to a second optical fibre for monitoring DO levels of a cell culture. The optical fibre terminator may be a triplex fibre terminator for terminating just three parallel optical fibres. For example, a triplex fibre terminator may connect to a first optical fibre for monitoring pH levels of a cell culture, to a second optical fibre for monitoring DO levels of a cell culture, and to a third optical fibre for monitoring pCO₂.

The optical fibre terminator may further include a sealing element configured to seal the terminator to a docking port. Conveniently, the sealing element may be located in a matching groove formed in the backshell of the terminator. For example, the sealing element may be an O-ring.

In a second aspect, the present invention provides a set (e.g. a pair or a trio) of terminated optical fibres terminated with the terminator according to the first aspect.

The polymer jackets of the optical fibres may be formed of polyethylene or polyurethane. Polyethylene and polyurethane are both soft plastics suitable for protecting the optical fibres from structural damage while allowing them to bend and flex as required.

Each of the terminated optical fibres may have a fibre diameter of 3 mm or less, and preferably of 2 mm or less. Advantageously, such a fibre diameter can ensure compatibility with small-scale bioreaction vessels.

A ratio of the centre-to-centre spacing between the end faces of the fibres at the predetermined spaced positions to the diameter of the fibres may be 2.3 or less, and preferably 2 or less, and more preferably 1.7 or less. Again, such a ratio can also ensure compatibility with small-scale bioreaction vessels.

The terminated optical fibres may further have a retention sleeve located rearwards of and spaced from the terminator and wrapping around the optical fibres. The sleeve can help to maintain the relative positions of the fibre set by the terminator for a distance behind of the terminator and thus help to prevent the fibres being accidently pulled apart in such a way that would damage them or the terminator, or cause the fibres end to shift relative to the end face.

In a third aspect, the present invention provides a combination of a screening system and plural sets (e.g. pairs or trios) of the terminated optical fibres according to the second aspect, wherein:
the screening system includes a plurality of sample wells for accommodating respective bioreaction vessels; and
each well includes a docking port and has the terminator of a respective set of the terminated optical fibres docked therein such that the optical fibres of the set can interrogate contents of a bioreaction vessel accommodated in the well via their fibre end faces.

Advantageously, such a screening system can enable screening of large numbers of potential cell clones in parallel cell-culturing processes to identify the most suitable clones for larger-scale development processes.

For example, the docking ports can be formed in the bases of the wells such that the end faces of the optical fibres are directed upwardly into the bioreaction vessels.

When the terminator of the first aspect includes a sealing element, each terminator may be sealed to its respective docking port via the respective sealing element. For example, the docking ports may have tapered profiles that progressively engage the sealing elements when the terminators are inserted therein. Advantageously, the sealing element can seal the optical fibres and parts of the screening system from spills from the bioreaction vessels, condensation and cleaning liquids.

The screening system may further include a temperature-controlled base on which the sample wells are supported, the optical fibres threading through apertures formed in the temperature-controlled base as they extend rearwardly away from the front faces of their terminators. For example, the temperature-controlled base may be formed of aluminium due to its high thermal conductivity.

In a fourth aspect, the present invention provides a method of assembling the combination according to the third aspect, the method including the steps of:
providing plural sets (e.g. pairs or trios) of the terminated optical fibres; and
threading trailing ends of each set of the terminated optical fibres through the docking port of the respective sample well before inserting the respective terminator into the docking port such that the optical fibres can interrogate contents of a bioreaction vessel accommodated in the well via their fibre end faces.

Advantageously, this convenient assembly method can be performed relatively quickly while also ensuring that the fibre end faces are correctly positioned to reliably interrogate contents of the bioreaction vessels. In addition, the trailing ends of the fibres can be suitably positioned for coupling to a sensing unit, e.g. a reader card for sending light signals into and reading light signals from the optical fibres.

When the combination of the third aspect is configured such that the screening system includes a temperature-controlled base, the method may further include a step of threading the trailing ends of the terminated optical fibres through the apertures formed in the base after the trailing ends have been threaded through the docking port.

The present invention includes combination of any of the aspects and optional features described, except where such a combination is clearly impermissible or expressly avoided.

### SUMMARY OF THE FIGURES

Embodiments of the invention will now be described by way of example with reference to the accompanying drawings in which:

FIG. 1 shows a perspective view of components of an optical fibre terminator according to an aspect of the present invention;

FIG. 2 shows a perspective side view of the optical fibre terminator of FIG. 1;

FIGS. 3A-3D show respectively a front elevation view, a side elevation view, a perspective view, and a plan view of a pair of terminated optical fibres terminated with the optical fibre terminator of FIG. 2;

FIG. 4 shows a partial plan view of a screening system;

FIG. 5 shows a plan view of a multi-well screening station of the screening system of FIG. 4;

FIG. 6 shows a sectional view of a well and a pair of terminated optical fibres of the screening system of FIG. 4;

FIG. 7 shows a perspective sectional view of part of the screening system of FIGS. 4 to 6

FIG. 8 shows a perspective view of components of a variant optical fibre terminator according to an aspect of the present invention;

FIG. 9 shows a perspective side view of the optical fibre terminator of FIG. 8;

FIGS. 10A-10D show respectively a front elevation view, a side elevation view, a perspective view, and a plan view of a pair of terminated optical fibres terminated with the optical fibre terminator of FIG. 9; and

FIGS. 11A-11C show plan views of the end faces of three possible triplex terminators.

### DETAILED DESCRIPTION OF THE INVENTION

Aspects and embodiments of the present invention will now be discussed with reference to the accompanying figures. Further aspects and embodiments will be apparent to those skilled in the art.

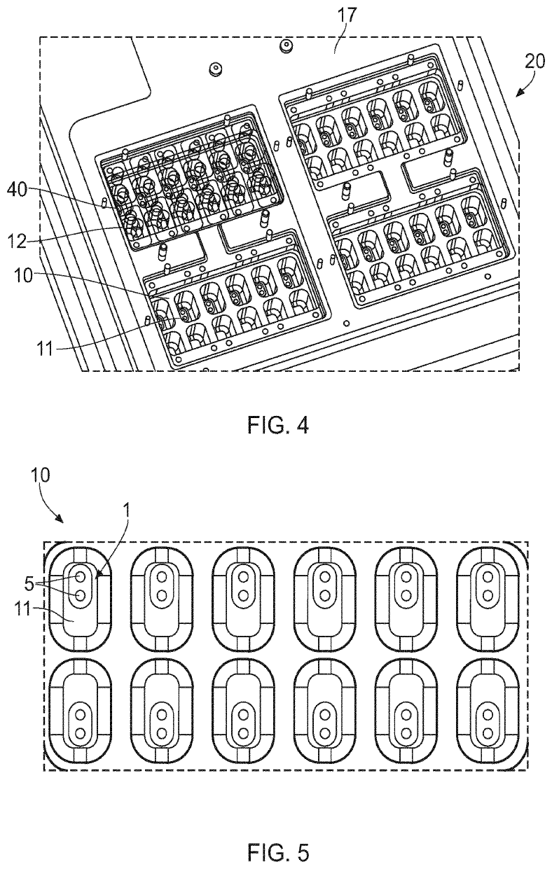

The present invention provides an optical fibre terminator for terminating optical fibres for non-invasive parameter monitoring of bioreaction contents contained in small-scale and closely spaced bioreaction vessels. The bioreaction vessels can be located in respective wells of a screening system discussed below in relation to FIGS. 4 to 6. In the following example the terminator is a duplex terminator for terminating just two parallel optical fibres. In particular, a first of the optical fibres can monitor pH levels of the bioreaction contents and a second of the optical fibres can monitor DO levels. The optical fibres have an optical waveguide core surrounded by a protective polymer jacket. Preferably, the fibres have a fibre diameter of 3 mm or less, and more preferably of 2 mm or less. Advantageously, such a fibre diameter ensures compatibility with small-scale and closely spaced bioreaction vessels.

The optical fibres can be commercially available fibres such as the Super Eska™ 2.0 mm Core Optical Fiber, 3 mm OD Polyethylene Jacket, V-2Y 1P1960/2000 manufactured by Mitsubishi Chemical Co. These fibres have polymer jackets formed of polyethylene and their outer diameter is 3 mm.

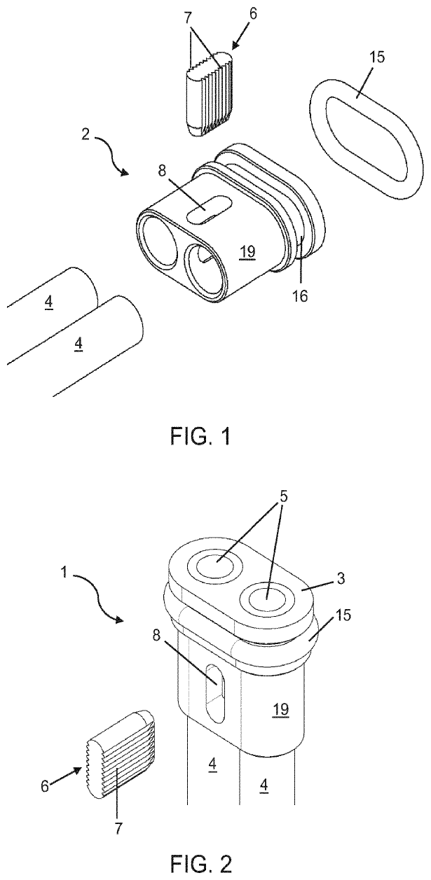

FIGS. 1 and 2 show perspective views respectively of (i) components of an optical fibre terminator 1 and (ii) the optical fibre terminator 1 as partially assembled. The terminator includes a housing 2 having a front face 3 (see FIG. 2) and a backshell 19 behind the front face. The front face includes respective openings to receive end faces 5 of the optical fibres 4. The terminator also includes a gripping spacer 6, the backshell including a channel 8 matching the shape of the gripping spacer such that the gripping spacer is removably insertable into the channel.

The housing in this example also includes a groove 16 formed behind the front face. The groove can accommodate a sealing element, such as an O-ring 15, for sealing the terminator to a docking port of the screening system discussed below in relation to FIGS. 4 to 6. The housing is configured for insertion thereinto of the two parallel optical fibres 4 so that the fibre end faces 5 are located at predetermined spaced positions relative to the front face 3 of the housing and the two fibres extend rearwardly away from the front face through the backshell of the housing. As shown in FIG. 2, when the fibre end faces are located at the predetermined spaced positions, they are flush with the front face. Additionally, a ratio of the centre-to-centre spacing between the end faces of the fibres at the predetermined spaced positions to the diameter of the fibres is 2.3 or less, preferably 2 or less and more preferably 1.7 or less. In this example, the centre-to-centre spacing between the end faces of the fibres is 4 mm and the ratio is 1.3. This ensures compatibility with small-scale and closely spaced bioreaction vessels.

Next, the gripping spacer 6 is inserted into the matching channel 8 such that it grips jacketed flanks of the two optical fibres 4 on opposite sides of the spacer to thereby space the fibres apart and retain the fibres in the housing 2 with the fibre end faces 5 locked flush with the housing end face 3. Advantageously, this reduces a risk of any unintentional withdrawal of the fibres from the housing and/or displacement of the fibre end faces from their predetermined spaced positions. The fibre retention is further enhanced by an obround cross-section of the spacer perpendicular to its insertion direction, the sides 7 of the spacer which grip the jacketed flanks of the two optical fibres 4 being the straight edges of the obround cross section. This configuration provides a relatively large contact area between the sides of the spacer and the jacketed flanks of the fibres to spread loads over the jacketed flanks and reduce risk of damage to the fibres. The opposite sides of the spacer are further serrated to increase the resistance to fibre withdrawal provided by the gripping spacer, e.g. by biting onto the jacketed flanks of the fibres, but advantageously without piercing polymer jackets. Overall, the gripping spacer firmly and safely retains the fibres within the housing while reducing the risk of structural damage to either the polymer jacket or the optical waveguide core.

The housing 2 of FIGS. 1 and 2 can be formed of a suitable plastic material such as polyether ether ketone (PEEK) or polyvinyl chloride (PVC). Advantageously, these materials exhibit low compressibility and can protect the fibres from damage due to external loads. Similarly, the gripping element 6 can be formed of a suitable plastic material such as a polycarbonate, polyether ether ketone (PEEK), or polyvinyl chloride (PVC). Advantageously, these materials are generally harder than the polymer jackets of the optical fibres, promoting good fibre retention inside the housing. The gripping spacer and the housing may be manufactured by any one of or any combination of: 3D printing, machining, and moulding.

According to another option, the gripping spacer 6 could be made of a rubber or elastomeric material that is pulled into the matching channel 8. The pulling stretches the material and causes it contract transversely so that, when released to relax to its unstretched state, it expands transversely to grip the jacket and retain it by frictional interaction therewith.

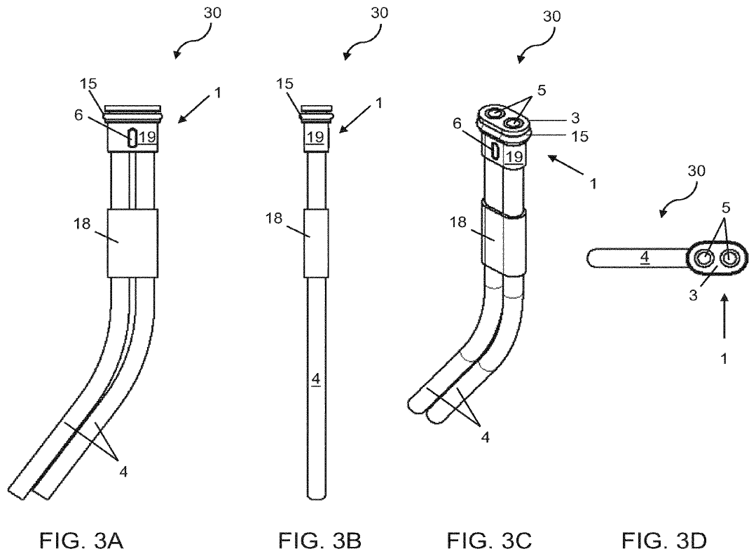

FIGS. 3A-3D show respectively a front elevation view, a side elevation view, a perspective view, and a plan view of a pair of terminated optical fibres 30. Identical features are referred to using the same reference numbers as in FIGS. 1 and 2. The terminated fibres of FIGS. 3A-3D additionally have a sleeve 18 located rearwards of the terminator 1 and wrapping around the optical fibres 4. The sleeve helps to maintain the fibre relative position introduced by the terminator 1 for a distance rearwards of the terminator and thus helps to prevent the fibres being accidently pulled apart in such a way that would damage them or the terminator, or cause the fibres end 5 to shift relative to the end face 3. Further rearwards of the sleeve, the fibres 4 can be more easily flexed, as shown in FIGS. 3A and 3C, thereby allowing the trailing ends of the fibres to be suitably positioned for coupling to a sensing unit, e.g. a reader card for sending light signals into and reading light signals from the optical fibres.

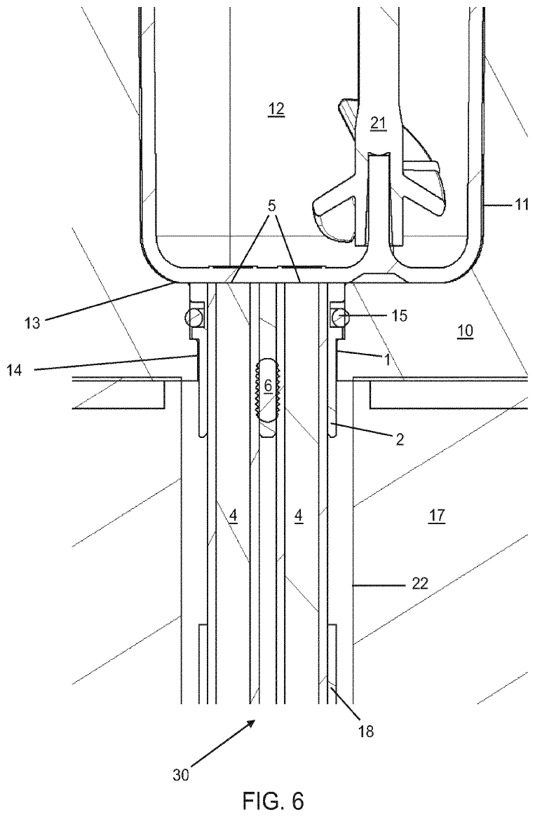

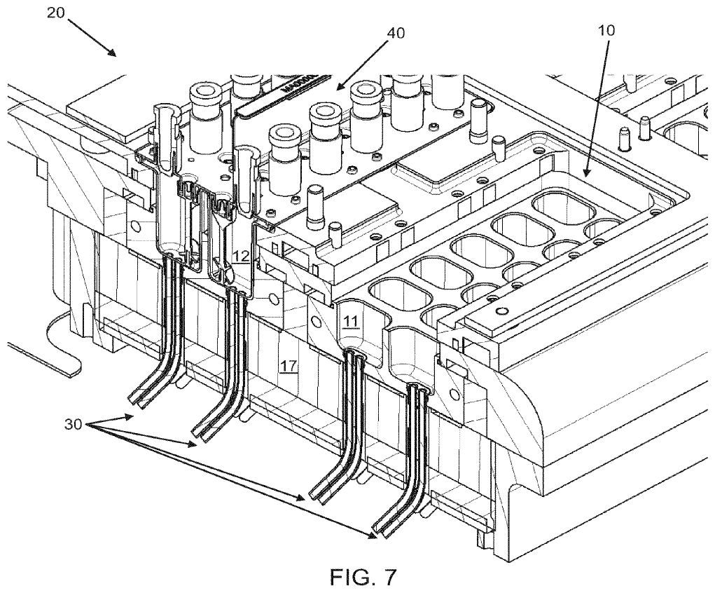

FIG. 4 shows a partial plan view of the screening system 20 including a plurality of sample wells 11 for accommodating respective bioreaction vessels 12, FIG. 5 shows a plan view of a multi-well screening station 10 of the screening system, FIG. 6 shows a sectional view of a well 11 and a pair of the terminated optical fibres 30 of the screening system, and FIG. 7 shows a perspective sectional view of part of the screening system.

Focusing initially on FIG. 4, the screening system 20 can enable screening of large numbers of potential cell clones in parallel cell-culturing processes to identify the most suitable clones for larger-scale development processes. The sample wells in this example are arranged in multi-well screening stations 10 (four of which are shown in FIG. 4), and the bioreaction vessels are similarly arranged in four, transparent plastic, bioreactor consumables 40 (only one of which is shown in FIG. 4). The bioreactor consumables and the multi-well screening stations have matching (in this case 2×6) layouts such that each well 11 accommodates a respective bioreaction vessel 12. The screening system includes a temperature-controlled base 17 (not shown in FIG. 4 but discussed below in relation to FIGS. 6 and 7) on which the wells are mounted. The base 17 in this example is formed of aluminium due to its high thermal conductivity. This can ensure that the temperature of the contents of the bioreactor consumables is reliably and accurately controlled.

Turning to FIG. 5, each multi-well screening station 10 includes twelve sample wells 11 arranged in two rows of six wells. Each well is connected to a pair of the terminated fibres 30 of FIGS. 3A-3D such that the fibre end faces 5 are directed upwardly into the respective bioreaction vessel 12 which, in use, is located in the well, as shown in FIG. 6. This can ensure a close contact between the fibre end faces and a base 13 of the respective bioreaction vessel, which can promote reliable interrogation of the contents of the bioreaction vessel by reducing a risk of compromised signal phase and amplitudes. This in turn helps to reduce a risk of loss of bioreaction control due to incorrect measurements. The screening system 20 of FIG. 4 has in total sixteen screening stations 10 requiring 16×12=192 pairs of terminated fibres. However, the optical fibre terminator 1 allows these pairs to be terminated quickly, accurately and reliably, and in a way that is compatible with fibre replacement, should that be needed.

The sectional view of FIG. 6 shows a single sample well 11 of one of the screening stations 10 mounted on the temperature-controlled base 17 and accommodating a single bioreaction vessel 12 of one of the bioreactor consumables 40. The vessel 12 has an agitator 21 for stirring and agitating its contents. The well has a base 13 and a docking port 14 formed in the base. The temperature-controlled base includes an aperture underneath each docking port leading to a channel 22 through the base. To mount a pair of the terminated optical fibres 30 to the well, trailing ends of the fibres are threaded through the docking port and then through the channel in the base. The terminator 1 is then docked in the docking port to secure the fibre end faces 5 such that they can reliably interrogate the contents of the bioreaction vessel. The O-ring 15 seals the terminator to the docking port to prevent accidental spills from the bioreaction vessel, condensation or cleaning liquids penetrating through the docking port. To improve the seal of the terminator to the docking port 14, the docking port may have a tapered profile that progressively engages the O-ring when the terminator is inserted therein.

As shown in FIG. 7, the trailing ends of the terminated optical fibres 30 extend completely through the channels 22 formed in the temperature-controlled base 17 to project from the underside of the base. At this position, the trailing ends can be coupled a reader card to facilitate sending light signals into and reading light signals from the optical fibres. For example, a commercially available reader card such as the Presens™ pH and DO reader card can be used. The reader card can be indexed from fibre to fibre to send to and read from each bioreaction vessel, whereby a single card can interrogate all the bioreaction vessels 12.

To replace individual malfunctioning or damaged fibres, first the respective bioreactor consumable 40 is removed from the screening station 10 to expose the pair of terminated fibres 30. Then, the fibres' trailing ends are pushed from underneath the screening system to cause the terminator 1 to protrude out of the well 11. This allows the terminator to be grasped and the pair of terminated fibres pulled out of the well. Once access to the terminator has been provided, the gripping spacer 6 can be removed and a selected fibre 4 can be withdrawn from the housing and replaced so that an end face of the replacement fibre is positioned at its predetermined spaced position relative to the other fibre and the housing end face, and the gripping spacer can be re-inserted to retain the fibres. Finally, the new pair of terminated fibres 30 can be connected to the screening station 20 in same manner as described in relation to FIG. 6.

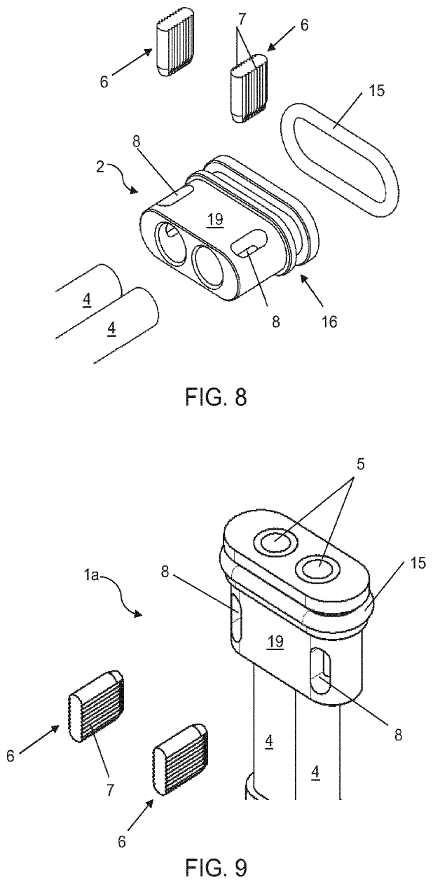

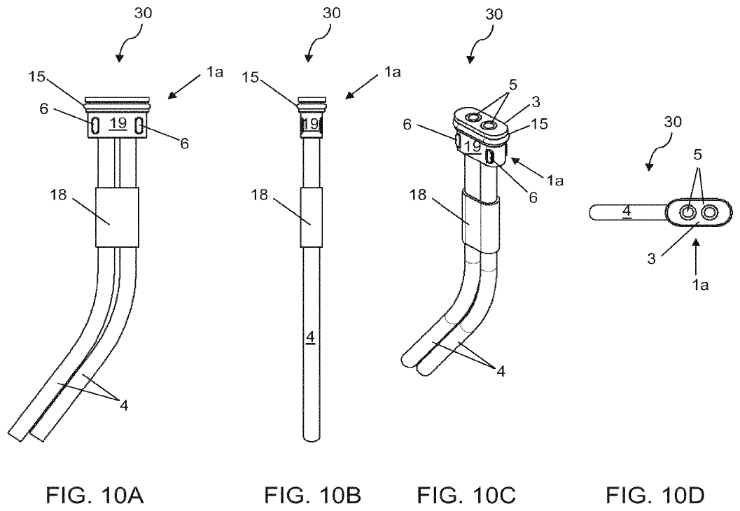

FIGS. 8 and 9 show perspective views respectively of (i) components of a variant optical fibre terminator 1a and (ii) the variant optical fibre terminator 1a as partially assembled. In addition, FIGS. 10A-10D show respectively a front elevation view, a side elevation view, a perspective view, and a plan view of a pair of optical fibres 30 terminated by the variant optical fibre terminator 1a. In FIGS. 8 to 10, features of the variant optical fibre terminator corresponding to those of the optical fibre terminator of FIGS. 1 to 7 are designated with the same reference numbers.

The variant optical fibre terminator 1a differs from that shown in FIGS. 1 to 7 by having two gripping spacers 6, and two matching channels 8 formed in the backshell 19 at opposite sides of the predetermined adjacent positions occupied by the optical fibres 4. When the gripping spacers 6 are inserted into their matching channels 8, one side of each grips the jacketed flank of the nearest optical fibre 4 to thereby press the fibres together and retain the fibres in the housing 2 with the fibre end faces 5 locked flush with the housing end face 3. The fibres can either be pressed together into direct contact with each other, or the housing may have a separator (not shown) which interposes between the two fibres to impose a fixed spacing on them as they press against opposite sides of the separator.

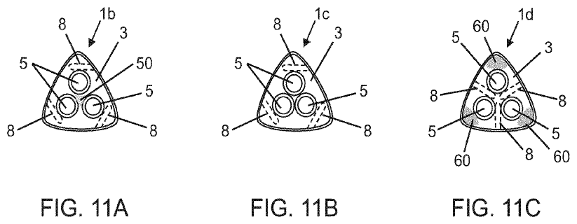

The terminators 1, 1a of FIGS. 1 to 10 are duplex terminators. However, the present invention can also be applied to terminators have more than two parallel optical fibres. For example, a triplex terminator may be provided having a trio of parallel optical fibres for monitoring respectively pH, DO and pCO₂. FIGS. 11A-11C show plan views of the end faces 3 of three such possible triplex terminators 1b, 1c, 1d. In the terminator 1b of FIG. 11A, the fibres are arranged such that their end faces 5 are centred on the corners of an equilateral triangle. Three channels 8 (their positions indicated by dashed lines) can receive three gripping spacers which press the fibres inwardly against a central separator 50 (its position indicated by a grey triangle) which extends rearwardly from the end face 3. In the terminator 1c of FIG. 11B, the same arrangement of channels 8 is adopted except that there is no central separator, and the fibres are pressed inwardly into direct contact with each other. In the terminator 1d of FIG. 11C, rather than having the gripping spacers press the fibres inwardly, the channels 8 are arranged so that the fibres are pressed outwardly against internal abutments 60 (indicated by grey zones) of the housing.

Advantageously, the terminators 1, 1a-1d of FIGS. 1 to 11 are easy to assemble, and require no gluing or bonding to connect to the optical fibres, thereby facilitating replacement of individual damaged or malfunctioning fibres. Moreover, the terminators do not require the polymer jackets of the fibres to be pierced in order to grip the fibres, and therefore reduce the risk of structural damage to the optical waveguide cores. Rather, the terminators can firmly and safely retain the fibres via the gripping spacer(s). Thus, the terminators reduce costs and increase reliability compared to conventional fibre attachment solutions discussed above. Additionally, the terminators can be made compact and are thus useable even with small-scale and closely spaced bioreaction vessels.

The features disclosed in the description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value.

When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. An optical fibre terminator for terminating two or more parallel optical fibres, each optical fibre comprising an optical waveguide core surrounded by a protective polymer jacket, the terminator including:
a housing having a front face and a backshell, and being configured for insertion thereinto of the two or more parallel optical fibres so that respective end faces of the two or more optical fibres are located at predetermined spaced positions relative to the front face and the two or more optical fibres extend rearwardly away from the front face through the backshell of the housing; and
one or more gripping spacers subsequently insertable into one or more respective, matching channels formed in the backshell behind the front face such that each gripping spacer, on its insertion, non-piercingly grips a jacketed flank of at least one optical fibre on a side of the spacer to thereby retain the fibres in the housing with the fibre end faces locked at the predetermined spaced positions.

2. The optical fibre terminator according to claim 1, which is for terminating two of the optical fibres, wherein:
a gripping spacer is insertable into a matching channel is formed in the backshell behind the front face and between the predetermined spaced positions such that the gripping spacer, on its insertion, grips jacketed flanks of the two optical fibres on opposite sides of the spacer to thereby space the fibres apart and retain the fibres in the housing with the fibre end faces locked at the predetermined spaced positions.

3. The optical fibre terminator according to claim 2, wherein the gripping spacer has serrated opposite sides to grip the jacketed flanks of the fibres.

4. The optical fibre terminator according to claim 1, which is for terminating two of the optical fibres, wherein:
two gripping spaces are insertable into respective matching channels formed in the backshell behind the front face and to substantially opposing sides of the predetermined adjacent positions such that each gripping spacer, on its insertion, grips a jacketed flank of a respective one of the optical fibres to thereby urge the fibres together and retain the fibres in the housing with the fibre end faces locked at the predetermined adjacent positions.

5. The optical fibre terminator according to claim 4, wherein each gripping spacer has a serrated side to grip the jacketed flank of the respective fibre.

6. The optical fibre terminator according to claim 1, wherein the or each gripping spacer is removably insertable into the housing.

7. The optical fibre terminator according to claim 1, wherein the or each gripping spacer has an obround cross section perpendicular to its insertion direction, the sides of the spacer(s) which grip the jacketed flanks of the optical fibres being the straight edges of the or each obround cross section.

8. The optical fibre terminator according to claim 1, wherein the front face of the housing includes respective openings to receive the fibre end faces and to define the predetermined spaced positions.

9. The optical fibre terminator according to claim 1, wherein when the fibre end faces are located at the predetermined spaced positions, the fibre end faces are flush with the front face of the housing.

10. The optical fibre terminator according to claim 1 which is a duplex fibre terminator for terminating just two parallel optical fibres, or a triplex fibre terminator for terminating just three parallel optical fibres.

11. The optical fibre terminator according to claim 1 further including a sealing element configured to seal the terminator to a docking port, wherein the sealing element is located in a matching groove formed in the backshell of the terminator.

12. A set of terminated optical fibres terminated with the terminator according to claim 11.

13. The set of terminated optical fibres according to claim 12, wherein the polymer jackets are formed of polyethylene.

14. The set of terminated optical fibres according to claim 12, wherein each of the terminated optical fibres has a fibre diameter of 3 mm or less, and preferably of 2 mm or less.

15. The pair of terminated optical fibres according to claim 12, wherein a ratio of the centre-to-centre spacing between the end faces of the fibres at the predetermined spaced positions to the diameter of the fibres is 2.3 or less, preferably 2 or less, and more preferably 1.7 or less.

16. A combination of a screening system and plural sets of the terminated optical fibres according to claim 12, wherein:
the screening system includes a plurality of sample wells for accommodating respective bioreaction vessels; and
each well includes a docking port and has the terminator of a respective set of the terminated optical fibres docked therein such that the optical fibres of the set can interrogate contents of a bioreaction vessel accommodated in the well via their fibre end faces.

17. The combination according to claim 16, wherein:
each terminator is sealed to its respective docking port via the respective sealing element.

18. The combination according to claim 16, wherein the screening system further includes a temperature-controlled base on which the sample wells are supported, the optical fibres threading through apertures formed in the temperature-controlled base as they extend rearwardly away from the front faces of their terminators.

19. A method of assembling the combination according to claim 18, the method including the steps of:
providing plural sets of the terminated optical fibres; and
threading trailing ends of each set of the terminated optical fibres through the docking port of the respective sample well before inserting the respective terminator into the docking port such that the optical fibres can interrogate contents of a bioreaction vessel accommodated in the well via their fibre end faces.

20. The method according to claim 19, the method further including a step of threading the trailing ends of the terminated optical fibres through the apertures formed in the temperature-controlled base after the trailing ends have been threaded through the docking ports.
